(19) United States
(12) Patent Application Publication
(10) Pub. No.: US 2016/0066827 A1
(43) Pub. Date: Mar. 10, 2016

(54) PULSE OXIMETRY RING

(71) Applicant: OWLET PROTECTION ENTERPRISES, LLC, Provo, UT (US)

(72) Inventors: Kurt Gibbons Workman, Provo, UT (US); Zachary David Bomsta, Provo, UT (US)

(21) Appl. No.: 14/849,366

(22) Filed: Sep. 9, 2015

Related U.S. Application Data
(60) Provisional application No. 62/048,086, filed on Sep. 9, 2014.

(51) Int. Cl. A61B 5/1455; A61B 5/00

(52) U.S. Cl. CPC A61B 5/14552; A61B 5/0002; A61B 5/6802; A61B 5/742

(57) ABSTRACT

A biometric sensor can be disposed within a wearable finger ring. The ring device can provide a variety of different biometric and health measurements. The ring device can also be in communication with the Internet. Information from the ring device can be saved in the cloud, accessed by remote smart devices, accessed by remote computers, or otherwise accessed and saved. Various users, such as doctors and EMS personnel, can access the information provided by the ring device.

### CROSS-REFERENCE TO RELATED APPLICATIONS

This application claims priority to and the benefit of U.S. Provisional Application Ser. No. 62/048,086, entitled “Pulse Oximetry Ring” filed on Sep. 9, 2014. The entire content of the above application is incorporated herein by reference in its entirety.

### BACKGROUND OF THE INVENTION

1. Technical Field

Implementations of the present invention relate to wearable devices that automatically track user health metrics.

2. Background and Relevant Art

Advancements in medical and wearable technology have given insight into the value of constantly monitoring and recording biometric and activity data. Patient monitoring and self-tracking can play an important role in the improvement and efficiency of healthcare and health outcomes. There are several products on the market that aim to accomplish constant monitoring of various metrics; however, incomplete feature sets and poor design-in terms of mobility, comfort and practical everyday use-prevent many individuals from benefitting from the improved health and lifestyle outcomes that can be achieved through constant biometric and activity monitoring.

Current market examples can be categorized as, but are not limited to, inconvenient pulse oximeters with wires and cords that prevent mobility, finger clip pulse oximeters, which impede everyday functions by attaching to the forefinger, and activity trackers that give limited biometric data. Activity trackers that are often worn on the wrist don't give sufficient insight because reliable biometric data such as heart rate and oxygen cannot easily be obtained accurately on the wrist and are therefore unreliable for acute monitoring. Other existing solutions, including home distress monitors, do not give sufficient insight into the overall health of the patient and simply act as an alert system with no proactive measures to improve or change health outcomes before an emergency event happens.

According, there are a number of problems in the art that can be addressed.

### BRIEF SUMMARY OF THE INVENTION

Implementations of the present invention comprise systems, methods, and apparatus configured to provide users with biometric and health tracking within a convenient and accurate package. In particular, implementations of the present invention comprise a ring device that a user can wear. The ring device can provide the user with various biometrics (e.g., heart rate, blood oxygen level, skin temperature, etc.) and various health measures (e.g., fall detection, sleep pattern recognition, movement tracking, etc.). Additionally, the ring device can be integrated into a broader system of devices that include Internet connectivity. As such, the ring device can also communicate biometric and health information to remote computers and users through the Internet.

Implementations of the present invention include a system for wirelessly monitoring the health of a user. The system can comprise a sensing module integrated within a wearable finger ring. At least a portion of the sensing module can be in contact with a user's finger. The sensing module can include a processing unit configured to receive and process health readings received by the sensing module. A wireless transmitter can also be in communication with the processing unit. The wireless transmitter can be configured to transmit the processed health readings to a base station. The base station can produce an alert if the processed health readings indicate a health trend that falls outside of a particular threshold.

Additional features and advantages of exemplary implementations of the invention will be set forth in the description which follows, and in part will be obvious from the description, or may be learned by the practice of such exemplary implementations. The features and advantages of such implementations may be realized and obtained by means of the instruments and combinations particularly pointed out in the appended claims. These and other features will become more fully apparent from the following description and appended claims, or may be learned by the practice of such exemplary implementations as set forth hereinafter.

### BRIEF DESCRIPTION OF THE DRAWINGS

In order to describe the manner in which the above recited and other advantages and features of the invention can be obtained, a more particular description of the invention briefly described above will be rendered by reference to specific embodiments thereof, which are illustrated in the appended drawings. Understanding that these drawings depict only typical embodiments of the invention and are not therefore to be considered to be limiting of its scope, the invention will be described and explained with additional specificity and detail through the use of the accompanying drawings in which:

FIG. 1 depicts various implementations of a ring device;

FIG. 2 depicts various additional implementations of a ring device;

FIG. 3 depicts yet more additional implementations of a ring device; and

FIG. 4 depicts a decision chart in accordance with an implementation of the present invention.

### DETAILED DESCRIPTION OF THE PREFERRED EMBODIMENTS

The present invention extends to systems, methods, and apparatus configured to provide users with biometric and health tracking within a convenient and accurate package. In particular, implementations of the present invention comprise a ring device that a user can wear. The ring device can provide the user with various biometrics (e.g., heart rate, blood oxygen level, skin temperature, etc.) and various health measures (e.g., fall detection, sleep pattern recognition, movement tracking, etc.). Additionally, the ring device can be integrated into a broader system of devices that include Internet connectivity. As such, the ring device can also communicate biometric and health information to remote computers and users through the Internet.

Accordingly, implementations of the present invention can be used in a variety of different settings and can provide significant benefits over the prior art. For example, in at least one implementation, a ring device can be used to improve health outcomes for patients leaving hospital. In particular, in light of new initiatives in healthcare reform that call for better health outcomes after hospital care. The ring device can provide hospital caregivers with near real-time patient health information. The ring device can be used at any point in the patient recovery process from intensive care to recovery to outpatient home care. In outpatient home care, the ring device can provide information that can be used to follow up with patients' health and activity after their stay in the hospital.

Additionally, in at least one implementation, the ring device can generate reports and even push automatic reminders to a patient. For example, a nurse can remotely program the ring device to periodically remind the patient to take a medicine. Similarly, in a case where a patient heart rate is being monitored, the ring device can be configured to automatically generate a heart rate report and to transmit it to the proper remote computer.

Similarly, the ring device can provide significant benefits and advantages in nursing home and home care settings. For example, many nursing homes neglect to use monitoring equipment with patients because it restricts mobility and a normal lifestyle. In contrast, using the ring device, caregivers can easily use remote monitoring tools without affecting lifestyle. In at least one implementation, the ring device can even replace existing call button equipment by integrating an alert button into the ring device itself. Similar benefits can be provided to home care settings where a patient can be remotely monitored in a way that does not interfere with the patient's lifestyle.

In addition to providing healthcare related monitoring, in at least one implementation, the ring device can also provide benefits within the recreation and athletic fields. For example, often high-altitude hikers desire to check their oxygen levels and heart rate. In at least one implementation, a ring device can allow the hikers to see an overall trend in the quality of their workout as well as their body's response to the journey. Similarly, a ring device can be used to improve almost any exercise regime by allowing a user to accurately and easily track his or her exercise routine.

In addition to the above benefits, the ring device can be of particular use in situations that make the use of conventional, bulky pulse oximetry difficult or impossible. For example, many small plane pilots use pulse oximeters to monitor their oxygen levels. The ring device can allow them to do this more comfortably using a significantly smaller form factor.

Similar benefits can be provided to the field of pediatrics. Most conventional pulse oximeters use adhesive leads or fingertip leads that restrict movement. It's hard to get a young child to want to leave this on his or her finger. In at least one implementation, the ring device can be disguised under a child friendly ring for an overall better patient experience.

Similar benefits can be provided within general hospital care. For example, conventional hospital acute monitoring consists of a bulky processing unit and chords running everywhere in the room. In at least one implementation, the ring device can remove the cords, send the data to the cloud, and also increase mobility from room to room.

In addition to the standard pulse oximeter monitoring, data can be collected about normal movement and activity. These trends could predict or pre-curse more serious problems such as a stroke. For example, sudden violent movements may be indicative of a seizure for some patients. Similarly, an extended lack of movement may be indicative of a loss of consciousness for some patients.

Additionally, in at least one implementation, the ring device can provide significant benefits within military operations. For example, the ring device could enable leaders to determine their soldiers response to certain events in the field. Gathering and analyzing the data could determine which soldiers are suitable for combat and which soldiers pose a threat to the team and operation. Additionally, medical Response and Emergency Medical Services (EMS) in the military is crucial. Seconds make the difference between life and death. Having military personnel outfitted with a ring device would give emergency response and medics the data they need to act quickly and save lives.

Further, in at least one implementation, the ring device can be used in conventional EMS operations. For instance, EMS response teams traditionally use fingertip oximeters on site but once the patient is transferred to the vehicle they hook them up to a traditional pulse oximeter with a bulky box, wires and chords. Space is limited in these vehicles and the ability to simply put a ring on the patient on site and have a seamless transition to the vehicle with no need to hook them up or use bulky monitors would provide significant benefits.

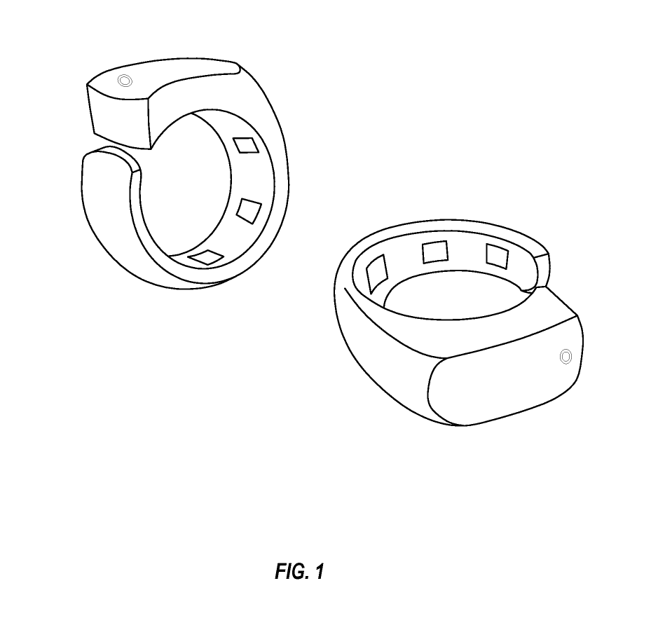

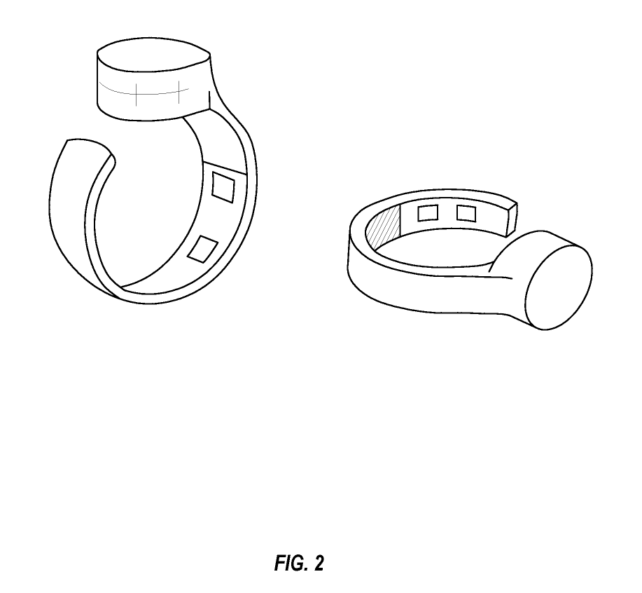

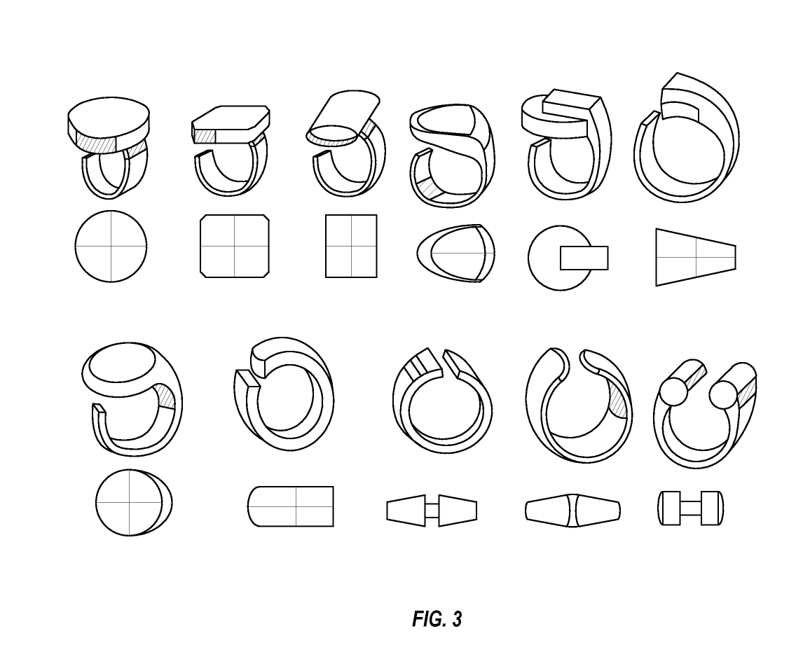

FIGS. 1-3 depict various implementations of a ring device. In at least one implementation, the ring device can comprise of a fitted form factor for greater style and durability. This form factor would be similar to a traditional ring where standard finger sizes would be manufactured and available, the caregiver or patient would simply choose a size that fit well on the least invasive finger. In at least one alternative implementation, other ring device designs include an adjustable ring that would allow for fewer variations to manufacturing, reducing cost and complexity. This implementation may also simplify the process in hospitals and stores where the device would initially be chosen. Various adjustable designs are depicted within FIGS. 1-3 For example, one implementation can include a circular “ring” with an open area in the circle, built with flexible material so as to be able to stretch and fit over different sizes of fingers.

The ring device can be implemented in a variety of different ways. For example, digital signal processing of light can be used to determine the heart rate of the individual using a well-known technology called pulse oximetry. Heart rate can be an important measurement in determining health, safety and emotion. Various implementations of sampling for blood oxygen levels can be implemented to reduce battery life such as sampling every 30 seconds versus constant monitoring.

In at least one implementation, using multiple photo diodes and LED lights, it is possible to increase the amount of light received into the processing unit and increase the DC signal to get better and more accurate pulse oximetry readings. Similarly, various unique combinations of the multiple photo diodes and LEDs can also be used to determine an ideal configuration. For example, a particular LED, or collections of LEDs, and a particular photo diode, or collection of photo diodes, may provide an optimum reading configuration. This configuration can be automatically determined by iterating through various configurations available on a particular ring device, until a desired configuration is obtained.

In addition to providing pulse oximetry data, in at least one implementation, the ring device can also provide movement data. For example, the ring device may comprise various accelerometers and gyroscopes that can be used to determine the normal activity of the patient and compare that to the current activity detected on the sensor. It can then be inferred how well the patient is doing, generally. Trends can raise awareness for family and caregivers that action may need to be taken. Similarly in a hospital, movement is also an important metric to measure the recovery and health of the patient. In at least one implementation, the ring device measures movement electrically and can send that information wirelessly.

Additionally, as disclosed above, in at least one implementation, the ring device can track sleep patterns. In particular, through heart rate and movement algorithms, data can be derived about the amount and the quality of a person's sleep. This can be valuable in helping them understand the best sleep environment and notify caregivers if a patient's sleep is suffering. Good sleep is tied to good health.

In at least one implementation, the ring device can also detect a user's position. For example, the ring may be configured such that it has an accelerometer and/or gyroscope that can determine when a user is positioned face down. Additionally, using the accelerometer, the product may be able to detect a “fall” event and send an emergency notification to caregivers and family members.

Implementations of the ring device may also be able to determine the temperature of a user's skin and the ambient temperature around the ring device using one or more electronic thermometers (e.g., thermistors). Additionally, in at least one implementation, the ring device may be able to interpolate a user's core temperature based upon the measured skin temperature and ambient temperature. The detected and interpolated information can also be reported wirelessly and used to sound alarms if certain thresholds are exceeded.

In addition to comprising a ring device, various implementations of the present invention can comprise base stations, routers, charging units, communication units, and other integrated or distinct components. For example, the charging unit may comprise a wireless charging pad that is configured to wirelessly charge the ring device. This can significantly increase the ease of use and ease of manufacture for a ring device.

Additionally, in at least one implementation, the ring device can also perform a form of proximity detection. In particular, the ring device and/or the base station may measure the signal strength from the wireless radio. Proximity and location could then be determined with the help of additional units within a home or living space. This would allow caregivers and family members to determine which room the patient is spending the most time in.

In at least one implementation, the ring device may also comprise a distress button. For example, the unit could be equipped with a distress button or something similar in case they need to call for help. The distress signal can then be sent through the phone or home Wi-Fi to caregivers and family members. The same system could replace the need for a call button in the hospital.

In at least one implementation, the ring device may communicate with a base station. The base station may further be in communication with the Internet through a wireless router. Alternatively, in at least one implementation, the ring device can communicate directly with a smart phone. As such, the smart phone can be used as an intermediary in communicating with the Internet. The ring device can communicate to the Internet and store data within the cloud.

The ring device can communicate to external devices (e.g., the base station) through a variety of different protocols. For example, Zigbee, Zwave, Bluetooth, 915 Mhz, Wifi, NFC are all protocols that can be used to connect the ring device to the base station or phone. The ring device can utilize various transmission paths to reach the Internet. For example, the ring device can communicate wirelessly to a base station that can communicate to a cloud service. Information in the cloud service can then be accessible by a smart device or computer. Additionally, in at least one implementation, the ring device can communicate wirelessly (e.g., with BLUETOOTH) directly to a smart device or computer.

Additionally, in at least one implementation, the base station can comprise a temperature sensor. This temperature sensor can monitor room or air temperature for comfort. This can also allow correlation to skin temperature to monitor core body temperature.

In at least one implementation, the base station can comprise an intercom. The intercom can be accessed remotely to allow caregivers to respond immediately through audio to let the patient know that help was on the way and give further instructions. Similarly, the intercom can provide automatic reminders and directions. For example, the base station and the ring can monitor the use of the ring device and remind the patient to put on their ring. Many people in their late ages forget to use devices like this, an automatic reminder could help form a habit and remind the patient.

The ring device may also comprise a display portion that provides various information and signals to a user. For example, simple LED lights can be used to indicate a variety of things from emergency alerts to poor signal or disconnection. A screen can also provide notifications and alerts as a method of preventing injury or death for example, a warning that heart rate is too high. These could include audible alerts or visual alerts to the patient or caregiver.

In at least one implementation, software can also be included within the ring device. The software can perform various data logging functions. The software can be provided to the user for local execution and/or may be installed on a remote server. The software can allow for data to be logged and stored on servers for analysis, generating reports and real time remote monitoring.

Additionally, in at least one implementation, the ring device, the base station, an associated smart device, or some other device may provide real-time alerts based upon received readings. In particular, real time alerts can be incorporated everywhere from the ring itself, the server, the app and any other hardware possibly incorporated in the unit. If a person's biometric or activity levels are out of range and instant alert or notification can be given not only to the person or patient but also to caregivers, family and friends.

While the ring device can provide significant benefits within health monitoring and healthcare scenarios, in at least one implementation, the ring device can also provide significant benefits in fitness and exercise scenarios. For example, because the device is connected and mobile it can provide the opportunity to gamify, engaging the patient and helping him/her reach desired goals set personally or by a caregiver. This gamification can take place on a mobile device but would be driven by physical behavior using the ring device as a translator between physical behavior and a digital interface. For example, a user can challenge a friend to meet certain health outcomes, as measured by the ring device. The user and friend can track each other through the use of respective ring devices. Gamification is used in a variety of industries to improve health outcomes, gather information and is even used as a reminder system. The gamification could even be used as a way to collect and analyze the patients desire and ability to improve health outcomes possibly being used as a better predictive insurance model based on behavior and desire to stay healthy.

Gathering information from all of the sensors listed above gives the ring device software the ability to look for correlations and use cross analytics to build a more predictive health model for each patient. Gathering and analyzing this data across thousands of individuals could prove predictive in trends, behavior and outcomes. This data has never been collected in this way or on this scale. At least one implementation can include a patient-monitoring platform purchased by hospitals to record and monitor all of their patients in one place. This would be accessible by mobile devices so Doctors and Caregivers can access the data anywhere and send real time advice and instructions to patients. The ring device connected to a platform would also allow caregivers and patients to communicate around real time data.

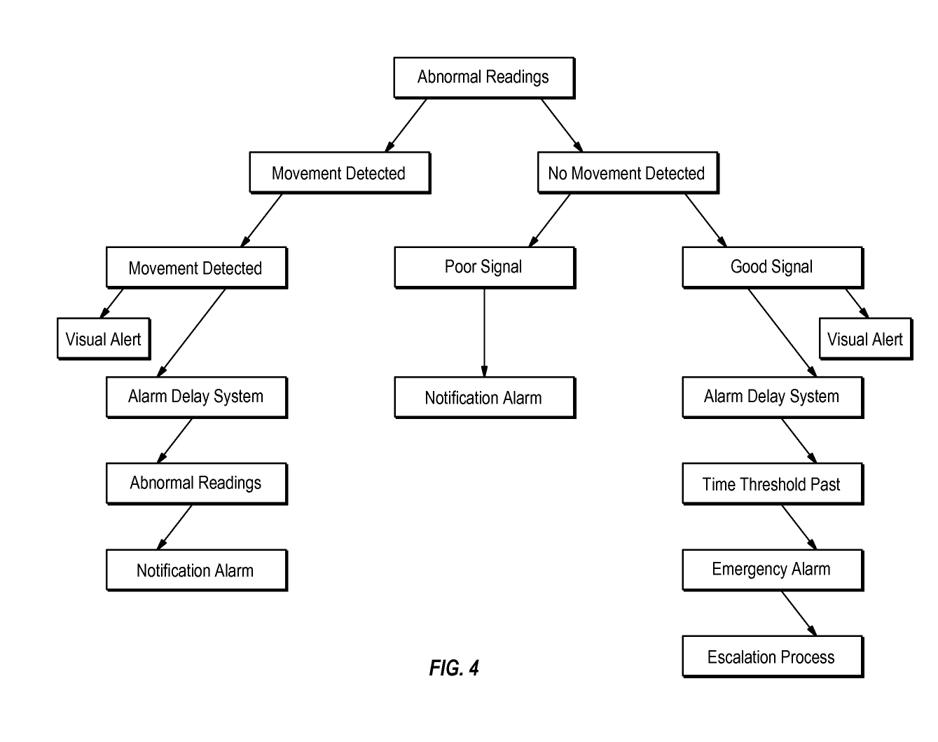

FIG. 4 depicts a logical flow chart associated with implementations of the present invention. In particular, FIG. 4 depicts a logical tree chart that describes an implementation of the logic that can be applied to an abnormal reading of health data by software at a ring device, base station, smart device, or any other computing device associated with the present invention. In general, an abnormal reading can consist of low oxygen levels or oxygen levels above realistic values such as 100 and above. Additionally, abnormal readings also can represent a heart rate that is too high or too low. Abnormal readings can also comprise a fall detection, irregular movement, irregular core temperature reading, or other abnormal biometric and/or health readings. Further, abnormal readings can also consist of an absence of a pulse oximeter reading or a bad pulse waveform. One will understand, however, that these are just potential reasons that an abnormal reading can occur, and are not meant as an exhaustive list of the abnormalities that the present invention can identify and/or compensate for.

Returning to FIG. 4, in block “a” an abnormal reading is detected. In at least one implementation, upon detecting an abnormal pulse oximetry reading, the software can determine if an accelerometer that is in contact with the user is detecting motion. If movement is detected (block b−1), the abnormal reading can be moved to a lower priority, because a moving user is less likely to have a dangerously low oxygen level and movement may be a primary cause of abnormal readings. In response to detecting motion, the software can generate a visual indication that an abnormal reading is being received, but that motion is being detected. For example, the software can generate a message on a remote computing device (e.g., smart phone, computer, etc.), or the software can display a visual indication on the base station.

In the case that abnormal readings continue, even if the software detects motion, the software can enter an alarm delay mode (block d−1). In particular, the software can allow for a threshold amount of time for the health readings to return to a normal level. The amount of the threshold can depend upon the detected oxygen level and the upward or downward trend. If after the threshold amount of time passes, the alarm is still producing abnormal readings (block e−1) then the software can raise the priority of the abnormal readings and signal an alarm. For example, the software can send an indication for an audible alarm (block f−1) on the remote computing device or the receiving station. This alarm can be a different alarm than the alarm used for a verified health concern. In particular, this alarm can be used to signify that the user may need attention, but is not likely in danger of a detected health issue.

Returning now to box “a” in FIG. 4. If the software detects abnormal readings and no movement is detected, the software can determine the strength of the pulse oximeter signal. For example, the software can determine that the signal from the pulse oximeter sensor is weak (block c−2), that the actual pulse oximeter readings are unreadable, or that the pulse oximeter readings are questionable because of outside influences. If the software determines that the signal is weak then the software can indicate that an alarm be sounded (block d−2). In at least one implementation, however, this alarm is different from the alarm that is sounded if a health concern is detected. Specifically, this alarm can be reserved for situation where a user may need attention, but it is not a health emergency.

In contrast, the software can determine that the signal from the pulse oximeter sensor is a readable signal (block c−3), or in other words, that the waveform and that SpO2 levels are clearly determined and the wireless signal is good. In this case, the software can indicate that a visual alert should be displayed. Similar to the visual alert described above, this alert can be displayed, for example, within a message on a remote computing device or within the display of the base station.

Also similar to above, the software can wait a threshold amount of time to determine if the health data returns to normal (block d−3). If the threshold time passes and the health readings do not return to a normal range (block e−3), the software can indicate that an emergency alarm be indicated (block f−3). In at least one implementation, this alarm is the highest priority alarm. Specifically, this alarm may be the loudest alarm and comprise a distinct sound.

In at least one implementation, this alarm may comprise an escalation process (block g−3). In particular, if the software does not detect a response to the alarm, the software can alert third parties, such as emergency responders or other designated individuals or devices. In at least one implementation of the present invention, a user can customize the priority and alarm that is associated with each of the above situation. For example, a user can specify that no alarm or indication be presented when abnormal readings are accompanied with the detection of motion.

Although the subject matter has been described in language specific to structural features and/or methodological acts, it is to be understood that the subject matter defined in the appended claims is not necessarily limited to the described features or acts described above, or the order of the acts described above. Rather, the described features and acts are disclosed as example forms of implementing the claims.

Embodiments of the present invention may comprise or utilize a special-purpose or general-purpose computer system that includes computer hardware, such as, for example, one or more processors and system memory, as discussed in greater detail below. Embodiments within the scope of the present invention also include physical and other computer-readable media for carrying or storing computer-executable instructions and/or data structures. Such computer-readable media can be any available media that can be accessed by a general-purpose or special-purpose computer system. Computer-readable media that store computer-executable instructions and/or data structures are computer storage media. Computer-readable media that carry computer-executable instructions and/or data structures are transmission media. Thus, by way of example, and not limitation, embodiments of the invention can comprise at least two distinctly different kinds of computer-readable media: computer storage media and transmission media.

Computer storage media are physical storage media that store computer-executable instructions and/or data structures. Physical storage media include computer hardware, such as RAM, ROM, EEPROM, solid state drives (“SSDs”), flash memory, phase-change memory (“PCM”), optical disk storage, magnetic disk storage or other magnetic storage devices, or any other hardware storage device(s) which can be used to store program code in the form of computer-executable instructions or data structures, which can be accessed and executed by a general-purpose or special-purpose computer system to implement the disclosed functionality of the invention.

Transmission media can include a network and/or data links which can be used to carry program code in the form of computer-executable instructions or data structures, and which can be accessed by a general-purpose or special-purpose computer system. A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer system, the computer system may view the connection as transmission media. Combinations of the above should also be included within the scope of computer-readable media.

Further, upon reaching various computer system components, program code in the form of computer-executable instructions or data structures can be transferred automatically from transmission media to computer storage media (or vice versa). For example, computer-executable instructions or data structures received over a network or data link can be buffered in RAM within a network interface module (e.g., a “NIC”), and then eventually transferred to computer system RAM and/or to less volatile computer storage media at a computer system. Thus, it should be understood that computer storage media can be included in computer system components that also (or even primarily) utilize transmission media.

Computer-executable instructions comprise, for example, instructions and data which, when executed at one or more processors, cause a general-purpose computer system, special-purpose computer system, or special-purpose processing device to perform a certain function or group of functions. Computer-executable instructions may be, for example, binaries, intermediate format instructions such as assembly language, or even source code.

Those skilled in the art will appreciate that the invention may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, switches, and the like. The invention may also be practiced in distributed system environments where local and remote computer systems, which are linked (either by hardwired data links, wireless data links, or by a combination of hardwired and wireless data links) through a network, both perform tasks. As such, in a distributed system environment, a computer system may include a plurality of constituent computer systems. In a distributed system environment, program modules may be located in both local and remote memory storage devices.

Those skilled in the art will also appreciate that the invention may be practiced in a cloud-computing environment. Cloud computing environments may be distributed, although this is not required. When distributed, cloud computing environments may be distributed internationally within an organization and/or have components possessed across multiple organizations. In this description and the following claims, “cloud computing” is defined as a model for enabling on-demand network access to a shared pool of configurable computing resources (e.g., networks, servers, storage, applications, and services). The definition of “cloud computing” is not limited to any of the other numerous advantages that can be obtained from such a model when properly deployed.

A cloud-computing model can be composed of various characteristics, such as on-demand self-service, broad network access, resource pooling, rapid elasticity, measured service, and so forth. A cloud-computing model may also come in the form of various service models such as, for example, Software as a Service (“SaaS”), Platform as a Service (“PaaS”), and Infrastructure as a Service (“IaaS”). The cloud-computing model may also be deployed using different deployment models such as private cloud, community cloud, public cloud, hybrid cloud, and so forth.

Some embodiments, such as a cloud-computing environment, may comprise a system that includes one or more hosts that are each capable of running one or more virtual machines. During operation, virtual machines emulate an operational computing system, supporting an operating system and perhaps one or more other applications as well. In some embodiments, each host includes a hypervisor that emulates virtual resources for the virtual machines using physical resources that are abstracted from view of the virtual machines. The hypervisor also provides proper isolation between the virtual machines. Thus, from the perspective of any given virtual machine, the hypervisor provides the illusion that the virtual machine is interfacing with a physical resource, even though the virtual machine only interfaces with the appearance (e.g., a virtual resource) of a physical resource. Examples of physical resources including processing capacity, memory, disk space, network bandwidth, media drives, and so forth.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

We claim:

1. A personal biometric monitoring system, the system comprising:
a sensing module integrated into a wearable finger ring, at least a portion of the sensing module in contact with a user's finger;
the sensing module comprising a processing unit configured to receive and process health readings received by the sensing module;
a wireless transmitter in communication with the processing unit, the wireless transmitter configured to transmit the processed health readings to a base station; and
the base station comprising a display that is configured to depict the processed health readings.
